(19) United States
(12) Patent Application Publication
(10) Pub. No.: US 2019/0192586 A1
(43) Pub. Date: Jun. 27, 2019

(54) PROBIOTIC BACTERIAL STRAINS BELONGING TO THE GENUS BIFIDOBACTERIUM AND PROBIOTIC CELL EXTRACTS (PCES) THEREOF HAVING IMMUNOSTIMULATING PROPERTIES

(71) Applicant: BIOIMMUNIZER SA, Lugano (CH)

(72) Inventor: Giovanni MOGNA, Novara (IT)

(21) Appl. No.: 16/331,499

(22) PCT Filed: Sep. 8, 2017
(86) PCT No.: PCT/IB2017/055423

§ 371 Date: Mar. 7, 2019

(30) Foreign Application Priority Data
Sep. 8, 2016 (IT) 102016000091033

(51) Int. Cl. A61K 35/745; A61P 31/00; A61P 29/00; A61P 39/06; A61P 37/00

(52) U.S. Cl. CPC A61K 35/745; A61P 31/00; A61P 37/00; A61P 39/06; A61P 29/00

(57) ABSTRACT

The present invention relates to probiotic bacterial strains belonging to the genus Bifidobacterium and/or probiotic cell extracts (PCEs) thereof, having immuno stimulating, anti-inflammatory and antioxidant properties. Furthermore, the present invention relates to a composition comprising a mixture comprising or, alternatively, consisting of said probiotic bacterial strains belonging to the genus Bifidobacterium and/or probiotic cell extracts (PCEs) thereof, having immunostimulating, anti-inflammatory and antioxidant properties. Finally, the present invention relates to said composition for use in the preventive and/or curative treatment of (i) inflammatory diseases, (ii) viral, bacterial, fungal or protozoan diseases, (iii) infections, and (iv) flu, cold or fever.

The present invention relates to probiotic bacterial strains belonging to the genus Bifidobacterium and/or probiotic cell extracts (PCEs) thereof, having immunostimulating, anti-inflammatory and antioxidant properties. Furthermore, the present invention relates to a composition comprising a mixture comprising or, alternatively, consisting of said probiotic bacterial strains belonging to the genus Bifidobacterium and/or probiotic cell extracts (PCEs) thereof, having immunostimulating, anti-inflammatory and antioxidant properties. Finally, the present invention relates to said composition for use in the preventive and/or curative treatment of (i) inflammatory diseases, (ii) viral, bacterial, fungal or protozoan diseases, (iii) infections, and (iv) flu, cold or fever.

It is well known that the constant, countless aggressions originating both from the external environment (microbes, bacteria, viruses, among others) and generated inside the body (mediators produced in excess, free radicals, catabolic derivatives, etc.), to which the body is subjected have shown to cause, over time, a weakening if not a breakdown of the immune defences that normally enable it to resist these aggressions.

The innate/inflammatory defensive reaction against said aggressions is activated in responses to external pathogens or signals originating from damaged tissue.

It is well known that inflammation is an essential part of our body's immune response. It is our body's attempt to heal itself after an external insult, hence an attempt to defend itself against viruses, bacteria or external agents or an attempt to repair damaged tissues.

Without inflammation, wounds would persist and infections could become fatal.

However, inflammation can trigger problematic processes that are difficult to resolve and play a fundamental role in some chronic diseases.

For this reason, it is necessary to conduct research and develop products capable of resolving and preventing inflammation: preventing and/or treating inflammatory processes means resolving all the mechanisms triggered by external pathogenic agents that lead to infection in our body.

Monocytes/macrophages have a key role in the initiation and resolution of inflammation via different activation programmes. Peripheral blood monocytes are in fact not a homogeneous population, but rather differ in their phenotypes and functions and, moreover, once they have differentiated into macrophages they can adopt in vivo a variety of different phenotypes that depend on changes in the tissue microenvironment, exhibiting a continuum of different functional states.

Circulating blood monocytes can be divided into two large subpopulations: “patrolling monocytes” (CX3CR1^{high}CD14^{dm}CD16+) and “inflammatory monocytes” (CCR2highCD14highCD16−).

CD16+ monocytes arrive at tissues constitutively and have different phenotypic characteristics compared to CD16− monocytes, which move only when the tissue is inflamed: in fact, CD16− monocytes seem to be involved in the innate inflammatory response. In contrast, cells deriving from the CD16+ population are involved in tissue homeostasis, and differentiate into specialised resident macrophages such as Kupffer cells, osteoclasts and microglia.

In light of these apparent physiological-functional differences, the lack of one or more of these subpopulations will obviously provoke different responses in various types of pathologies.

The signals present in different microenvironments deriving from pathogenic agents such as viruses, bacteria, fungi or protozoa, and from tissue lesions, or effectors deriving from adaptive immunity, thus trigger different genetic programmes that lead monocytes to differentiate into macrophages following different functional states of polarisation.

The current paradigm describes two functional subsets: M1 macrophages, or macrophages activated in a classic manner, and M2 macrophages, activated in an alternative manner: it should be considered that these functional phenotypes represent the two extremes of a wide spectrum of states of differentiation.

Therefore, practitioners in the field continue to be highly interested in being able to intervene and thus modulate, in a useful and effective manner, the activity of monocyte/macrophage subpopulations in order to advantageously form a first line of defence against infections, for example viral and bacterial infections. In practical terms, there remains a need to be able to have a therapy/treatment which makes it possible to prevent and/or treat viral, bacterial, fungal or protozoan diseases rapidly and effectively, as well as to prevent and/or treat the inflammatory processes triggered by external pathogenic agents which lead to infection in the body.

After a long, intense research and development activity, the Applicant has devised an effective composition based on probiotic bacterial strains and/or probiotic cell extracts (PCEs) thereof, and an associated method for the preparation thereof, which is capable of modulating the activity of subpopulations of CD16− and CD16+ monocytes in such a way as to regulate their function in inflammatory diseases.

The present invention relates to probiotic bacterial strains belonging to the genus Bifidobacterium and/or probiotic cell extracts (PCEs) thereof with immunostimulating, anti-inflammatory and antioxidant properties, having the features as defined in the appended independent claim.

The present invention relates to a composition comprising a mixture comprising or, alternatively, consisting of probiotic bacterial strains belonging to the genus Bifidobacterium and/or probiotic cell extracts (PCEs) thereof with immunostimulating, anti-inflammatory and antioxidant properties, having the features as defined in the appended independent claim.

The present invention relates to a composition comprising a mixture comprising or, alternatively, consisting of probiotic bacterial strains belonging to the genus Bifidobacterium and/or probiotic cell extracts (PCEs) thereof with immunostimulating, anti-inflammatory and antioxidant properties, said composition being for use in the preventive and/or curative treatment of (i) inflammatory diseases, (ii) viral, bacterial, fungal or protozoan diseases and (iii) infections, having the features as defined in the appended independent claim.

The present invention relates to a method (or procedure) for the preparation of probiotic bacterial strains belonging to the genus Bifidobacterium and/or probiotic cell extracts (PCEs) thereof with immunostimulating, anti-inflammatory and antioxidant properties, having the features as defined in the appended independent claim.

The present invention relates to a method (or procedure) for the preparation of a mixture comprising or, alternatively, consisting of probiotic bacterial strains belonging to the genus Bifidobacterium and/or probiotic cell extracts (PCEs) thereof with immunostimulating, anti-inflammatory and antioxidant properties, said composition being for use in the preventive and/or curative treatment of (i) inflammatory diseases, (ii) viral, bacterial, fungal or protozoan diseases and (iii) infections, having the features as defined in the appended independent claim.

Preferred embodiments of the present invention are described below by way of example, thus without limiting the scope hereof.

In the context of the present invention, “probiotic cell extracts or probiotic bacteria cell extracts (abbreviated PCE)” is meant to include, without any limitation, also the cell wall extracts of the probiotic bacteria used (BCWEs) and/or the peptidoglycans that are a constituent component of the cell wall. The bacterial strains belong to the genus Bifidobacterium and are selected from among the following species: B. longum, B. breve, B. animalis susp. lactis, B.bifidum etc.

The composition of the present invention comprises a mixture and, optionally, additives and/or excipients and/or other ingredients, all the above being pharmaceutical or food grade.

Said mixture, contained in said composition of the present invention, comprises or, alternatively, consists of probiotic bacterial strains belonging to the genus Bifidobacterium and/or probiotic cell extracts (PCEs)—or bacterial cell wall extracts (BCWEs). Said extracts, contained in said mixture, comprise or, alternatively, consist of peptidoglycans.

Said mixture, contained in said composition of the present invention (which comprises probiotic bacterial strains belonging to the genus Bifidobacterium and/or probiotic cell extracts (PCEs)—or bacterial cell wall extracts (BCWEs) comprising or, alternatively, consisting of peptidoglycans) shows an immunostimulating action, an anti-inflammatory action and/or an anti-oxidant action which enable said composition to be used where the external agent (viruses, bacteria, fungi or protozoa) has induced the inflammation in order to be able to prevent and/or treat the inflammatory process induced by viruses, bacteria, fungi or protozoa.

Said mixture and, therefore, also said composition of the present invention (which comprises probiotic bacterial strains belonging to the genus Bifidobacterium and/or probiotic cell extracts (PCEs)—or bacterial cell wall extracts (BCWEs) comprising or, alternatively, consisting of peptidoglycans) are capable of preventing and/or treating the inflammatory processes triggered by external pathogenic agents (viruses, bacteria, fungi or protozoa) which lead to infection in the body.

In one embodiment, said mixture and, therefore, said composition comprise or, alternatively, consist of at least one probiotic bacterial strain belonging to the species B. longum or B. breve or B. animalis susp. lactis, or B.bifidum, or an extract of the corresponding probiotic cells (PCE).

In one embodiment, said mixture and, therefore, said composition comprise or, alternatively, consist of at least one probiotic bacterial strain belonging to the species B. longum or B. breve or B. animalis susp. lactis, or B.bifidum selected from among the strains B. animalis subsp. lactis BS01 (LMG P-21384), B. breve BR03 (DSM 16604) and B. longum BL03 (DSM 16603), or an extract of the corresponding probiotic cells (PCE).

The Applicant has studied and analysed in an in-depth and detailed manner the immunostimulating properties of a very vast group of bacterial strains, all belonging to the genus Bifidobacterium. In particular, bacterial strains belonging to the following species belonging to the genus Bifidobacterium: B. longum, B. breve, B. animalis susp. lactis, B.bifidum etc. were analysed and studied.

The bacterial strains were used at different states of viability: (i) viable in the exponential growth stage, (ii) tyndallized (Probiotical) and (iii) destroyed by sonication. Furthermore, for the same strains of probiotic bifidobacteria under examination, their respective probiotic cell extracts (PCEs) obtained through a specific extraction protocol better detailed below (Protocol P1 for the extraction of probiotic cell extracts (PCEs)) were also studied and analysed.

We specifically describe, by way of example, only the study of three different strains of Bifidobacteria (B. animalis subsp. lactis BS01 (LMG P-21384), B. breve BR03 (DSM 16604) and B. longum BL03 (DSM 16603)) which were used at different states of viability: (i) viable in the exponential growth stage, (ii) tyndallized (Probiotical) and (iii) destroyed by sonication, and their respective probiotic cell extracts (PCEs) or bacterial cell wall extracts (BCWEs) obtained through extraction Protocol P1.

The method (or procedure) devised by the Applicant and which is the subject matter of the present invention is described below.

The procedure applied by the Applicant comprises the application of a series of methods or protocols of analysis and evaluation. The procedure applied by the Applicant is described below and comprises or, alternatively, consists of:
Extraction of probiotic cell extracts (PCEs) of the above-mentioned probiotic bacterial strains of the different strains (PROTOCOL P1).
Isolation of the mononuclear cells (PBMCs) from peripheral blood of healthy subjects with standard protocol P2 (PROTOCOL P2).
Stimulation of the PBMCs with the strains and BCWEs (10 mg/ml) under examination for 24 h and 5 days (120 hours)-(STIMULATION METHOD).
Evaluation of ROS production.
Evaluation of the monocyte/macrophage differentiation markers.
Data analysis and statistical evaluation performed with a paired samples T-Test; the data are considered significant for values of p<0.05.

Protocol P1: EXTRACTION OF PCEs from the Different Strains of Bifidobacteria.

A cytofluorimetric analysis is performed in order to assess the cellular concentration of the starting lyophilisate with the commercial kit “BDTM cell viability kit with BD liquid counting beads” according to method ISO 19344:2015.

The results of the starting bacterial cell count are 2.8±0.3 MLD/mg (MEAN±STANDARD DEVIATION).

Materials
Strains of Bifidobacteria analysed: a animalis subsp. lactis BS01 (LMG P-21384), B. breve BR03 (DSM 16604) and B. longum BL03 (DSM 16603)).
Saturated solution of ammonium sulphate at T° 20 C.
Balance, Beaker, Graduated Cylinder, Mini Blender, Spatula, Glass Tubes, Magnetic Stirrer, Magnetic Stirring Rod, Pipette, Vortex, Centrifuge 900-10000 g.
Distilled or double-distilled water.

Protocol P1
Suspend 30 g of lyophilised Bifidobacteria in 120 ml of distilled or double-distilled H₂O (1 g of lyophilised bacteria in 4 ml of H₂O, dilution 1:4).
Stir with the magnetic stirrer and stirring rod until obtaining a homogeneous suspension.
Transfer the homogeneous bacterial suspension into the jar of the blender, making sure that the liquid does not exceed the level of the blender blades. For the purpose of crushing the bacterial wall it is important to maintain the liquid at the level of the blender blades. In the case of a Philips blender, 40 ml of suspension at a time is recommended.
Carry out 2 crushing cycles of 1 minute each, with a 1 minute pause in between. In order to decrease the temperature of the preparation add 1 teaspoon of crushed ice in the blender.
Collect the suspension in a beaker and repeat the operation until all the bacteria of the original suspension have been crushed.
Centrifuge the mixture at 900×g for 5 minutes in order to separate any still intact bacteria, which will form sediment (pellets), from the wall fragments, which will remain in the supernatant.
Collect the supernatant in a beaker, making sure to remove the sediment, and centrifuge again at 10000×g for 15 minutes (or at 6000×g for 20 minutes). The supernatant is collected and stored, preferably frozen, for further applications. This portion, which we call endomass, contains all of the cytoplasmic components of the bacterial cell. The peptidoglycan fractions are contained in the sediment.
Resuspend the pellets (containing the fractions of interest) in H₂O and reach a volume of about 90 ml.
The peptidoglycan fractions are then precipitated by adding, under stirring, a saturated solution of ammonium sulphate [(NH₄)₂SO₄] until 40% saturation.

$$\frac{X}{V + X} = \frac{40}{100} \qquad FORMULA$$

where:

X=amount of ammonium sulphate that must be used;

V=volume of the solution to be precipitated.

Therefore, for a V=90 ml, 60 ml of saturated ammonium sulphate will have to be added.
Place the suspension on a magnetic stirrer and add, dropwise, the saturated solution of ammonium sulphate (60 ml).
Place the solution at 4° C. overnight in such a way that precipitation of the peptidoglycan occurs.
Centrifuge the solution at 10000×g for 15 minutes (or at 6000×g for 20 minutes) in order to collect the precipitate.
Eliminate the supernatant containing the ammonium sulphate and repeat the washes 5 times [10000×g for 15 minutes (or at 6000×g for 20 minutes)]. All the ammonium sulphate still present in the peptidoglycan suspension will be eliminated.
Resuspend the precipitate in water and proceed to liophilise the peptidoglycan obtained.

A mean yield (Peptidoglycan/Lyophilisate) of 0.12 grams (Standard deviation 0.06) and a hexosamine level of 10.19% (Standard deviation 1.14) were obtained. The yield is the peptidoglycan fraction expressed as final grams of peptidoglycan (final product used for the experimentation)/grams of the starting lyophilisate. The data were obtained by averaging all of the batches of lyophilisates used in the experimentation. Whereas the hexosamine level present in the peptidoglycan fraction represents an indicator of the purity of the composition of the final product obtained. The final concentration of the peptidoglycan used in the experimentation and which demonstrated to have the maximum biological activity is equal to 10 mg/ml.

Protocol P2: Separation of the Peripheral Blood Mononuclear Cells.

The peripheral blood mononuclear cells (PBMC) were separated by density gradient centrifugation. For this purpose, in each experiment use was made of 20 ml of buffy coat from healthy donors admitted to the Immunotransfusion Centre of the Hospital of Borgomanero; a mean yield of 200×10⁶ PBMC/buffy was obtained. The quantity of separated cells was determined by means of a cell count in Bürker chambers, using Turk's solution, and enabled a distinction between mononuclear cells and polymorphonuclear cells. The cells were brought to a concentration of 2×10⁶ cells/ml in RPMI-1640 growth medium (Invitrogen) supplemented with 10% fetal bovine serum (FBS, Gibco), heat inactivated, 1% glutamine and 25 mM Hepes.

Stimulation Method: Stimulation of PBMCs with Bifidobacteria.

After separation, the PBMCs were stimulated with the bacterial strains (live, tyndallized and sonicated and respective BCWE) for 24 h and 5 days. The tests used were the following:
cytofluorimetric test to evaluate the specific surface markers of the monocyte/macrophage populations.
test to evaluate the production of ROS (oxygen free radicals) in an in vitro model of hyperhomocysteinaemia.

The internal controls for each single experiment were represented by:
LPS (lipopolysaccharide) 1 mg/ml for the evaluation of the monocyte/macrophage subpopulation. The cells were stimulated for 5 days and the cell surface markers specific for each individual subpopulation were evaluated through cytofluorimetric analysis. The final value is the expression of the fold increase in the population versus a positive control activated by LPS.
Homocysteine 5 mM for the test on ROS production. A spectrophotometer reading was taken at 550 nm after 24 h+2 h of stimulation. The Abs value read is directly proportional to the release of free radicals in the supernatant.

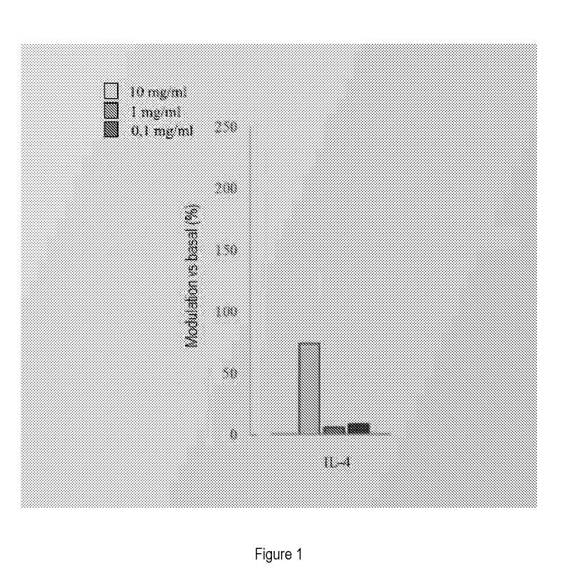

The optimal concentration of stimulation with BCWEs (FIG. 1 Modulation vs basal (100%)) was determined. From the results obtained it may be seen that the concentration of 10 mg/ml represents the optimal peptidoglycan concentration for modulating interleukin IL4, an important regulatory cytokine in the inflammatory process.

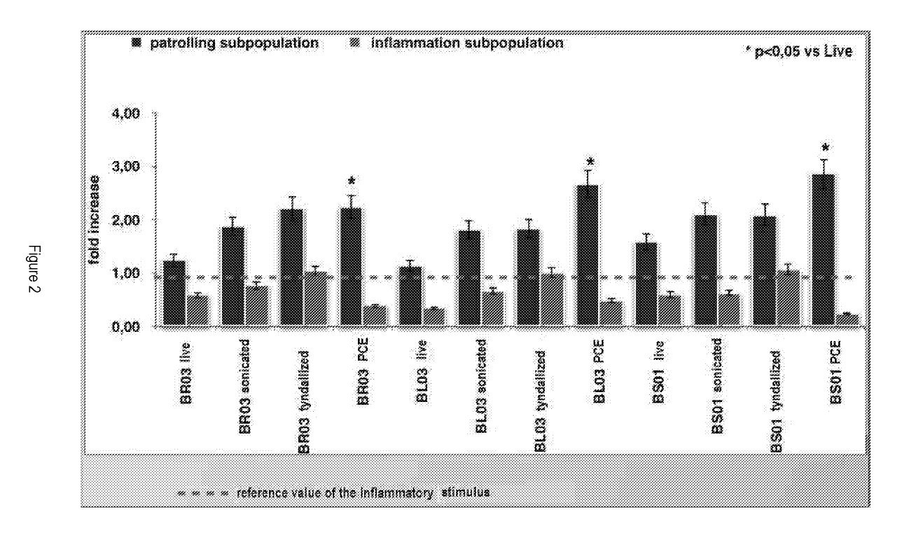

FIG. 2 shows the results of modulation in relation to the monocyte/macrophage population after 5 days of stimulation. The cytofluorimetric evaluation regards the surface markers.

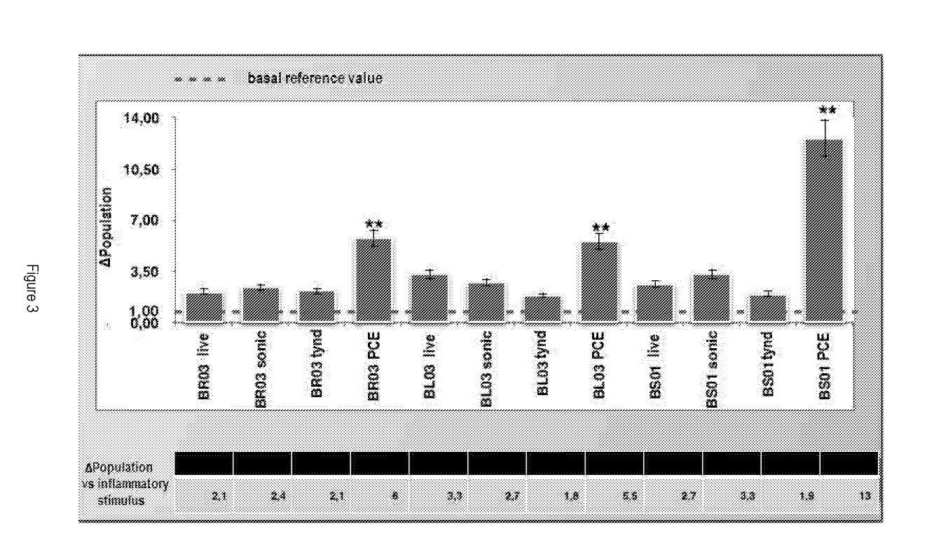

FIG. 3 shows the results regarding the activation ratio between the two monocyte/macrophage populations (CD16+/CD16−).

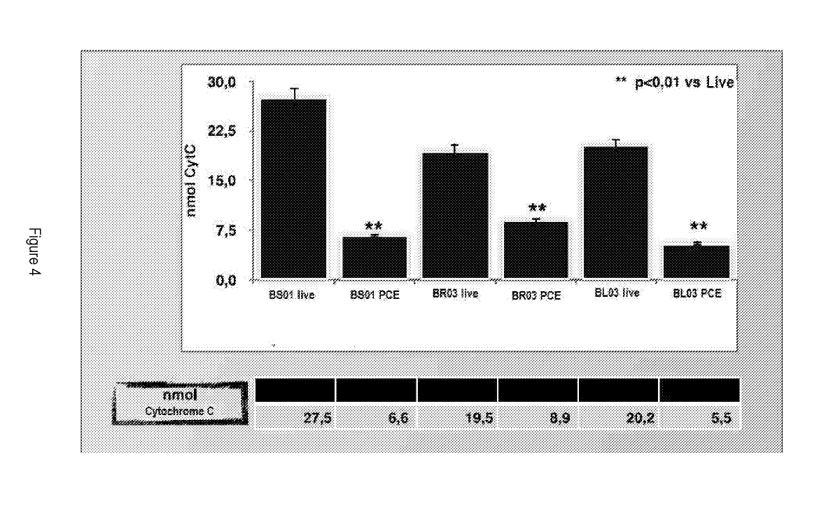

FIG. 4 shows the results of ROS production after 24 hours. With reference to FIG. 4 it should be borne in mind that:

CTR=basal value of ROS release: 8.9 nmol;

HYC=positive reference value, cells stimulated with homocysteine 5 mM: 42.1 nmol;

Every other point=24 h pre-stimulation with probiotics followed by 2 h of stimulation with homocysteine.

The experimentation (24 h+2 h) was followed by 30′ incubation with cytochrome C (1 mg/ml) and a spectrophotometer reading at 550 nm in order to have an indirect quantification of the production of oxygen free radicals.

BACTERIAL SONICATION: 3 ml of fresh culture broth (at a concentration of about 1×10⁹ bacteria/ml) are added to 27 ml of sterile water in a sterile test tube; the sample thus prepared is sonicated for 15 minutes. A cytofluorimetric analysis is performed to evaluate the residual cell concentration, using the commercial kit “BDTM cell viability kit with BD liquid counting beads” according to method ISO 19344:2015.

BACTERIAL TYNDALLIZATION: A cytofluorimetric analysis is performed to quantify the total concentration of the cells that are intact but not viable, using the commercial kit “BDTM cell viability kit with BD liquid counting beads” according to method ISO 19344:2015. The procedure applied for the tyndallization is described in Italian patent application no. M12012A001355 and in international patent application PCT/IB2015/058747.

The probiotic cell extracts (PCEs, comprising or, alternatively, consisting of peptidoglycans) generated by the three strains tested, all belonging to the genus Bifidobacterium, demonstrated to be capable of modulating, to a significant degree, the two human monocyte/macrophage subpopulations, both the “patrolling subpopulation” and the “inflammatory subpopulation”, thus showing a strong immunostimulating effect.

Monocytes/macrophages constitute a first line of defence in viral and bacterial infections. Furthermore, these cells are capable of regulating the immune response and play a primary role in inducing and maintaining the inflammatory process. The monocytes circulating in the peripheral bloodstream migrate into different tissues, where they differentiate into specialised cells as a result of exposure to different microenvironmental factors. The macrophages thus differentiated are present in various organs, where they play an important role in immune and inflammatory responses, tissue damage and repair, the clearance of apoptotic cells and antineoplastic surveillance.

As may be seen from FIG. 3, the probiotic cell extracts (PCEs, comprising or, alternatively, consisting of peptidoglycans) from the strain B. animalis subsp. lactis BS01 (LMG P-21384) bring about a 13-fold increase (basal value equal to 1) in the ratio between the two populations: this is an important indicator of the anti-inflammatory and immunostimulating activity typical of these two populations. In fact, by modulating the two subpopulations to a significant degree, the PCEs stimulate the specific functional immune characteristics of monocytes/macrophages, including the ability to phagocytise microorganisms (key role in innate immunity), the ability to process antigens and present them to T lymphocytes (key role in acquired immunity) and the ability to synthesize numerous cytokines and chemokines, which are capable of initiating and/or amplifying or ending an inflammatory reaction, promoting the recruitment of inflammatory cells and modulating the lymphocyte response.

The probiotic cell extracts (PCEs, comprising or, alternatively, consisting of peptidoglycans), by stimulating the two subpopulations, are capable of helping to combat inflammation in the infected site, and activating the adaptive response against pathogenic bacteria, viruses and fungi.

Advantageously, said mixture and, therefore, said composition comprises or, alternatively, consists of the probiotic strain B. animalis subsp. lactis BS01 (LMG P-21384) and/or a probiotic cell extract (PCE).

Furthermore, PCEs are capable of significantly reducing the oxidative stress induced by homocysteine (in vitro model of hyperhomocysteinaemia).

The action of the PCEs is better than that of the viable, tyndallized or sonicated strains of BR03, BS01 and BL03 and represents an important anti-oxidant and anti-inflammatory effect of PCEs.

Advantageously, the probiotic cell extracts (PCEs, comprising or, alternatively, consisting) can be validly applied in the preventive or curative treatment of infections thanks to their triple effect:
(a) immunostimulating, since peptidoglycans are capable of modulating the activation of the two monocyte/macrophage populations directly involved in the inflammatory response;
(b) anti-inflammatory, since peptidoglycans are capable of inducing a regression in the inflammatory state provoked by the presence of external pathogenic agents, such as, for example viruses, bacteria, fungi or protozoa;
(c) anti-oxidant, since peptidoglycans furthermore have a direct action in decreasing the oxidative stress triggered by the populations of the immune system directly involved in inflammation.

The present invention relates to the probiotic bacterial strains belonging to the genus Bifidobacterium and/or probiotic cell extracts (PCEs) thereof with immunostimulating, anti-inflammatory and antioxidant properties, for use in the preventive and/or curative treatment of flu, fever or cold, or for use as an anti-flu vaccine.

Advantageously, said vaccine comprises or, alternatively, consists of at least one strain of bacteria belonging to the following species belonging to the genus Bifidobacterium: B. longum, B. breve, B. animalis susp. lactis, B.bifidum; said strains are selected from among the strain B. animalis subsp. lactis BS01 (LMG P-21384), B. breve BR03 (DSM 16604) and B. longum BL03 (DSM 16603).

Said mixture and, therefore, said composition of the present invention have demonstrated to reinforce the immune system compared to live, lysed, tyndallized or sonicated cells of the same probiotic strains and to decrease the inflammatory impact. In practical terms, they are capable of (i) reducing, as a means of prevention (vaccine), the number of individuals who become ill, for example with flu, cold or fever; and/or (ii) reducing the intensity of the symptoms (fewer days of illness) and the severity thereof (for example, less fever). For this reason, the composition of the present invention is a valid alternative to the vaccines present on the market.

PCEs have a comparable, if not even better, immunomodulating activity in the case of activation of monocyte/macrophage subpopulations, when compared to the ability of the corresponding viable strain. It follows that the use of PCEs is a valid alternative to the use of the viable strains, or lysed strains, or tyndallized strains or sonicated strains, especially in formulations such as, for example, formulations for topical use (gels, creams, ointments and lotions) or formulations for oral use or for use in aerosol therapy. Said formulations can also be marketed in climate zones that would be unsuitable for live probiotic bacterial strains.

The Applicant conducted a further in vitro study.

In this study an analysis was made of the ability of the probiotic strain B. animalis subsp. lactis BS01 (LMG P-21384) and the respective cell wall extract thereof (PCE), obtained through the extraction process described in this patent application, to restore membrane integrity in a model of a damaged intestinal epithelium.

The following were performed:
1. Extraction of cell walls from the different strains under examination.
2. Trans Epithelial Electrical Resistance (TEER) assay.
3. The data analysis and statistical evaluation were performed with a paired-sample t-test; the data were considered significant in the case of values of p<0.05.

Materials and Methods

The probiotic strain B. animalis subsp. lactis BS01 (LMG P-21384) was cultured at 37° C. in a medium of election (MRS) and counted using a FACSCalibur flow cytometer (produced by BD). The strain was counted at a 10-3 dilution in physiological solution using the Cell Viability Kit of the company BD according to the standard procedure.

For the experimentation, CACO-2 cells were treated with 3×10⁶ bacteria and 10 mg/ml of PCEs.

The experimentation provided for the use of 24-well transwell plates, on which CACO-2 cells were cultured for 21 days until forming a confluent cell layer, in vitro model of intestinal epithelial tissue produced by the company ARETA International, Gerenzano (Va).

All experiments were performed in DMEM 10% FBS (fetal bovine serum) medium (GIBCO).

The protocol for evaluating the restoration of the barrier function envisages, after a reading of the initial resistance of each well, a pre-incubation of the CACO-2 cells for 1 h with pro-inflammatory stimuli: TNFalpha and IL-1beta (produced by Immunotools) at a final concentration of 10 ng/ml, which induce epithelial damage, followed by 1 h incubation with the probiotic strain (3×106 cells/well) and the respective PCE thereof (10 mg/ml).

After 2 h of stimulation, the medium in the apical chambers and in the basolateral chambers is replaced. 24 h after stimulation the TEER (Trans-Epithelial Electrical Resistance) is determined in the experimental cellular model using an EVOM2—instrument specifically designed to measure transepithelial resistance in cell tissues—which can perform a correct measurement of the electrical resistance between the apical chamber and the basolateral chamber.

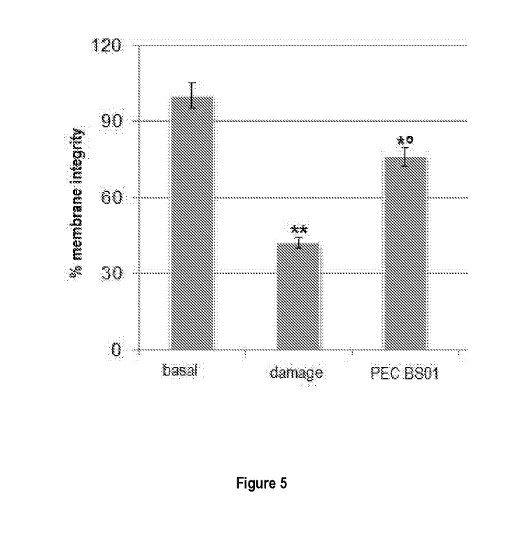

The TEER measurements are presented as a percentage of the TEER values for each individual experimental well in comparison with the values of the control well (basal value). The results are shown in Table 1 and FIG. 5.

TABLE 1

| | TEER | | |
|---|---|---|---|
| | mean % | st err | significance |
| basal | 1100 | 9 | |
| damage | 42 | 3.9 | p < 0.01 ** vs basal; |
| | | | p < 0.05 * vs basal; |
| PCE BS01 | 76 | 8.2 | p < 0.05 ° vs damage |

Discussion and Conclusions

Maintaining intestinal integrity is critical for the basal physiological processes of the intestine.

The integrity of the barrier can be measured through transepithelial electrical resistance, or TEER. TEER is an in vitro measurement of the passage of ions through paracellular pathways.

Therefore, a reduction in the TEER values can represent an early expression of cell damage and indicate that the barrier is compromised.

It has already been demonstrated that the probiotic strain B. animalis subsp. lactis BS01 (LMG P-21384) is in itself capable of restoring the integrity of damaged membrane in an intestinal epithelial cell model (CACO-2), by bringing the TEER value to 85%.

In this study the aim was to demonstrate that the BS01 strain maintains this ability even after the cell wall extraction process described in this patent application.

The data confirm that the PCEs of the BS01 strain restore membrane integrity to 76%, in a manner wholly comparable to that of the original strain.

In light of this evidence, it can be affirmed that the PCE of the BS01 strain is an excellent candidate for use in the prevention and the treatment of intestinal wall damage due to bacterial or viral infections.

1. Probiotic bacterial strains belonging to the genus Bifidobacterium and/or probiotic cell extracts (PCE) thereof with immunostimulating, anti-inflammatory and antioxidant properties, for use in the preventive and/or curative treatment of (i) inflammatory diseases, (ii) viral, bacterial, fungal or protozoan diseases, (iii) infections, and (iv) flu, cold or fever.

2. The bacterial strains for use according to claim 1, wherein said strains are selected from the group of bacterial strains belonging to the following species belonging to the genus Bifidobacterium: B. longum, B. breve, B. animalis susp. Lactis and B. bifidum.

3. The bacterial strains for use according to claim 1 or 2, wherein said strains are selected from among the bifidobacteria B. animalis subsp. lactis BS01 (LMG P-21384), B. breve BR03 (DSM 16604) and B. longum BL03 (DSM 16603).

4. A probiotic cell extract of bifidobacteria belonging to the genus Bidifobacterium selected from among the species B. longum, B. breve, B. animalis subsp. lactis, and B. bifidum, said extract comprising or, alternatively, consisting of peptidoglycans.

5. The extract according to claim 4, wherein the strains are selected from among the strains B. longum BL03 (DSM 16603), B. breve BR03 (DSM 16604) and B. animalis subsp. lactis BS01 (LMG P-21384).

6. A composition comprising a mixture comprising or, alternatively, consisting of probiotic bacterial strains belonging to the genus Bifidobacterium and/or probiotic cell extracts (PCE) thereof with immunostimulating, anti-inflammatory and antioxidant properties, according to any one of claims 1-5 and, optionally, a food or pharmacologically acceptable additive or excipient or ingredient; said composition being for use in the preventive and/or curative treatment of (i) inflammatory diseases, (ii) viral, bacterial, fungal or protozoan diseases, (iii) infections, and (iv) flu, cold or fever.
